(19) United States
(12) Patent Application Publication
(10) Pub. No.: US 2011/0253802 A1
(43) Pub. Date: Oct. 20, 2011

(54) TAN TENT

(76) Inventor: Michelle Elizabeth FREELOVE, Battle Creek, MI (US)

(21) Appl. No.: 13/090,045

(22) Filed: Apr. 19, 2011

Related U.S. Application Data
(60) Provisional application No. 61/325,559, filed on Apr. 19, 2010.

(51) Int. Cl. B05B 1/00

(52) U.S. Cl. 239/69

(57) ABSTRACT

A system for applying a tanning spray to a user comprising a tent apparatus, spray apparatus, and fluid directing apparatus is disclosed. The tent apparatus can contain a canopy, a fly, a groundsheet, a footprint, an entrance, and supports for holding the canopy. The spray apparatus can contain a nozzle, a nozzle holder, tubing connector, peripheral tubing, tube splitter, master tube, and a fluid directing device connector. The fluid directing device can contain a user interface, microprocessor and computer readable memory, a pump, motor, actuator, tanning fluid reservoir, and tanning fluid.

### CROSS REFERENCES

This application is a nonprovisional of 61/325,559 filed Apr. 19, 2010, FIGS. 1-5 are incorporated herein by reference.

### FIELD OF THE INVENTION

A system and method for applying sun tanning spray on a user.

### BACKGROUND OF THE INVENTION

US Patents applications: 2004/0232257; 2006/0231567; 2006/0118039; 2010/0288320; and U.S. Pat. Nos. 2,333,915; 4,582,062; 5,540,383; 5,790,992; 6,081,944; and 6,585,751 relate to tents, spraying systems, UV tanning systems, sprinklers, and treatment systems.

### SUMMARY OF THE INVENTION

None of the ten cited U.S. patents and patent applications provide a mechanism for applying tanning solution in a portable, space efficient, time efficient manner. To overcome these problems, a system for applying a tanning spray to a user comprising a tent apparatus, spray apparatus, and fluid directing apparatus is disclosed. The tent apparatus can contain a canopy, a fly, a groundsheet, a footprint, an entrance, and supports for holding the canopy. The spray apparatus can contain a nozzle, a nozzle holder, tubing connector, peripheral tubing, tube splitter, master tube, and a fluid directing device connector. The fluid directing device can contain a user interface, microprocessor and computer readable memory, a pump, motor, actuator, tanning fluid reservoir, and tanning fluid.

### BRIEF DESCRIPTION OF THE DRAWINGS

FIG. 1A shows two nozzle rows connected to a pitched tent apparatus and the fluid directing device.

FIG. 1B shows a tent apparatus with the entrance in the open position and the actuator on the groundsheet.

FIG. 2A shows an exploded view of a nozzle, nozzle holder, canopy piece, tubing connector and peripheral tube.

FIG. 2B shows an assembled view of a nozzle, nozzle holder, canopy piece, tubing connector and peripheral tube.

FIG. 2C shows a nozzle row having three peripheral tubes and three nozzles.

FIG. 2D shows two nozzle rows connected to a tent apparatus.

FIG. 3 shows a schematic view with the outer shell of the pump removed.

FIG. 4 illustrates a flow chart of the user assembling the tent apparatus.

FIG. 5 illustrates a flow chart of the user tanning in the tent apparatus.

FIG. 6 illustrates a flow chart of the user cleaning the tent apparatus.

### DETAILED DESCRIPTION OF THE INVENTION

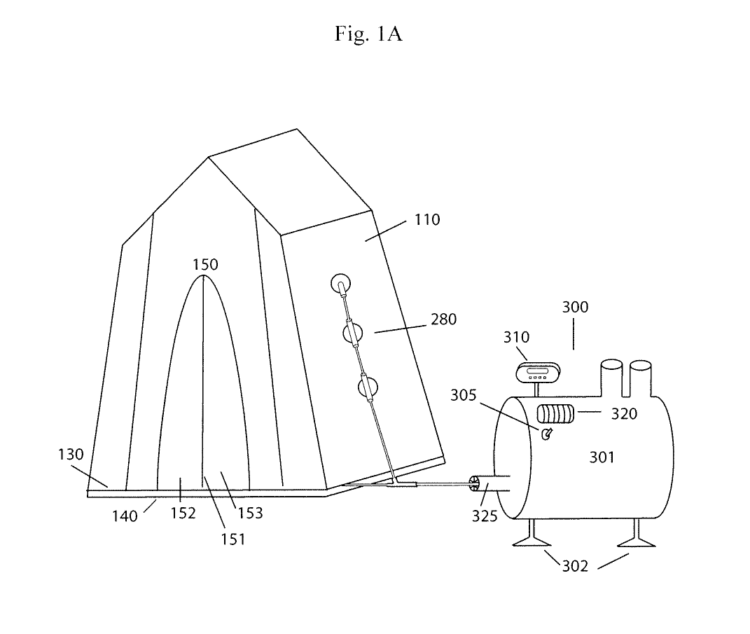

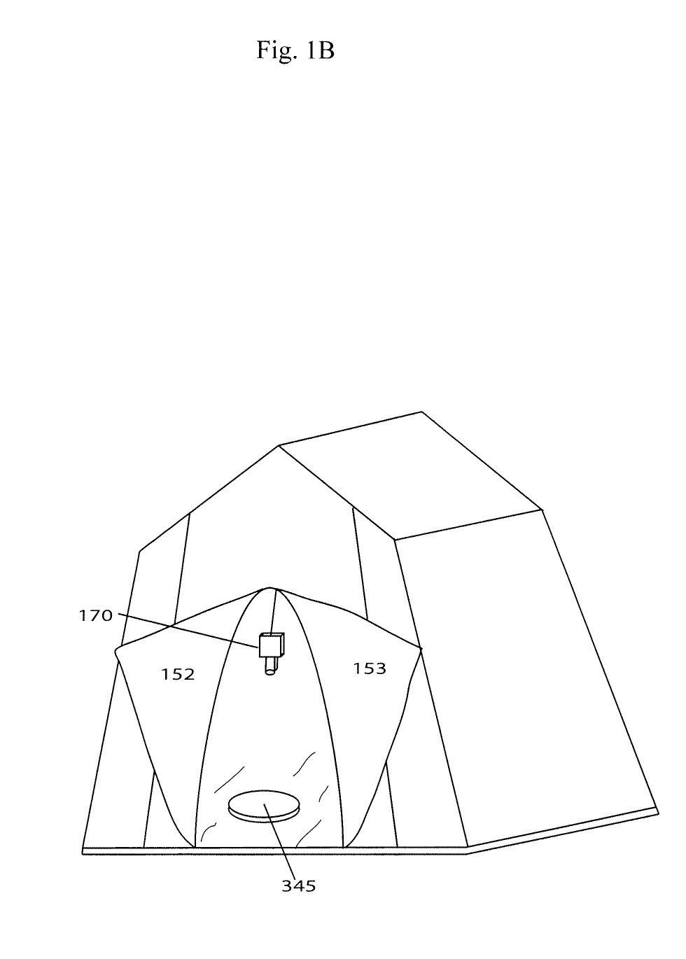

As shown in FIG. 1A, one aspect of the present invention provides a system 1 containing a tent apparatus 100, spray apparatus 200, and fluid directing device 300.

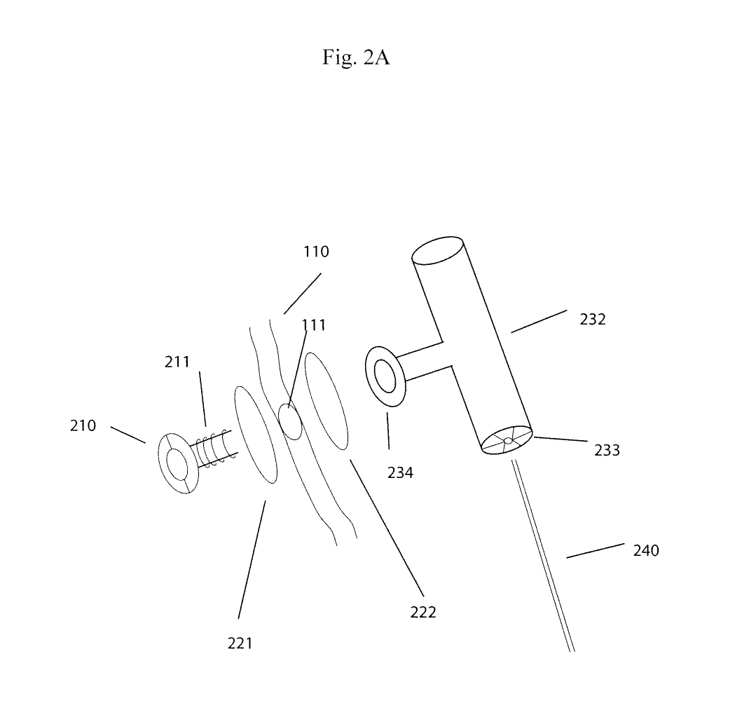

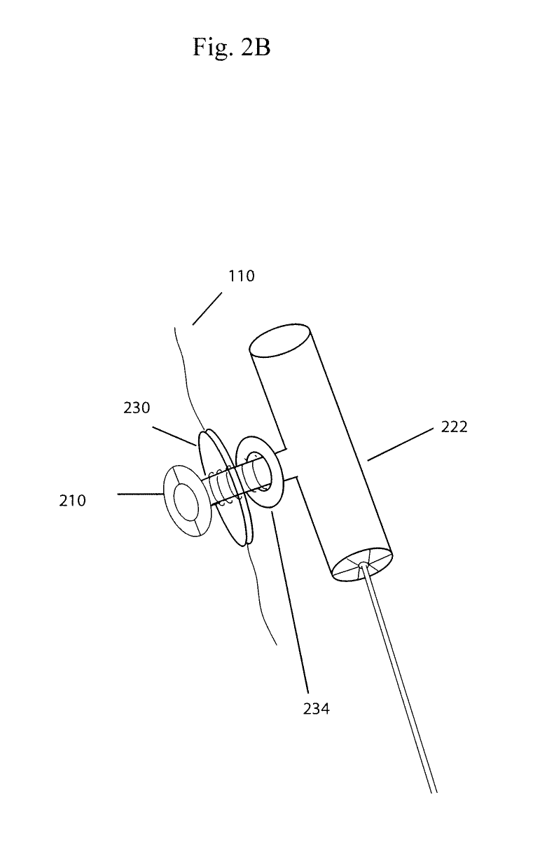

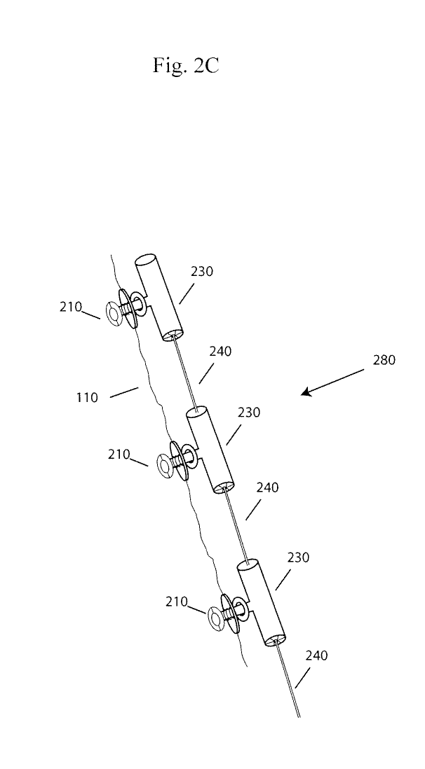

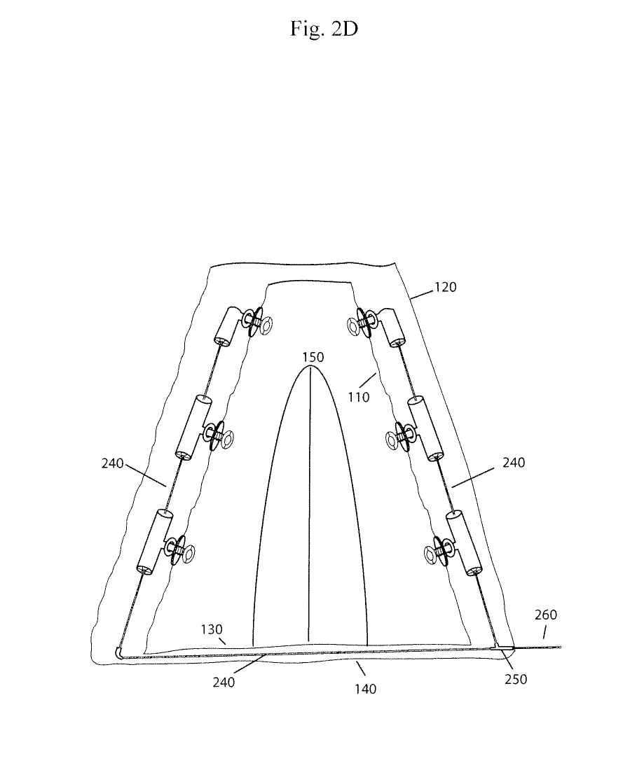

As shown in FIGS. 1A and 1B, the tent apparatus 100 may contain a canopy (inner tent) 110, a fly (outer tent) 120 (FIG. 2D), a groundsheet (inner bottom of the tent) 130, a footprint (out bottom of the tent) 140, an entrance (like a doorway) 150, supports 160 (such as poles) for holding the canopy, and a detecting apparatus 170. The tent may exist in a pitched (FIG. 1A) or unpitched position. The canopy 110 may contain holes 111, or the user may make holes 111 in the canopy to facilitate the mounting of nozzles (210) to the canopy 110. In some embodiments, the nozzles would be positioned to spray inside the tent, the tubing outside the canopy running along the sides and/or top of the tent apparatus, and covered by the fly 120. The entrance may have a zipper 151 in the middle to create two door flaps (152 and 153) on each side of the zipper.

As shown in FIGS. 2A-2D, the spray apparatus 200 may contain a nozzle 210, a nozzle holder 220 (the one shown in FIG. 2A has two pieces 221 and 222), tubing connector 230 (such as a single connector 231 or T-connector 232), peripheral tubing 240, tube splitter 250 (FIG. 2D), master tube 260, and a fluid directing device connector 325 (FIG. 1A). The nozzle may contain a threaded fastener 211 for insertion into the receptacle 234 of the tubing connector. The threaded fastener may pass through the hole 111 of the canopy 110, and be supported by the nozzle holder 220. The connectors may contain a valve 233 wherein a valve is engaged into the closed position when the connector and tube are disengaged. This arrangement allows tubes and connectors to be split without risking leaking tanning solution. The peripheral tubing may be connected to the tubing connectors. The tubing connectors can connect the tubing to the nozzles via the nozzle holder. The nozzle holder may contain a threaded center bore which receives the nozzle and the connector. The nozzle holder may be placed into a hole in the canopy, and contain an outer rim to protect the canopy. The nozzle holder may be removable, or it may be fixed to the canopy via soldering, glue, or stitching.

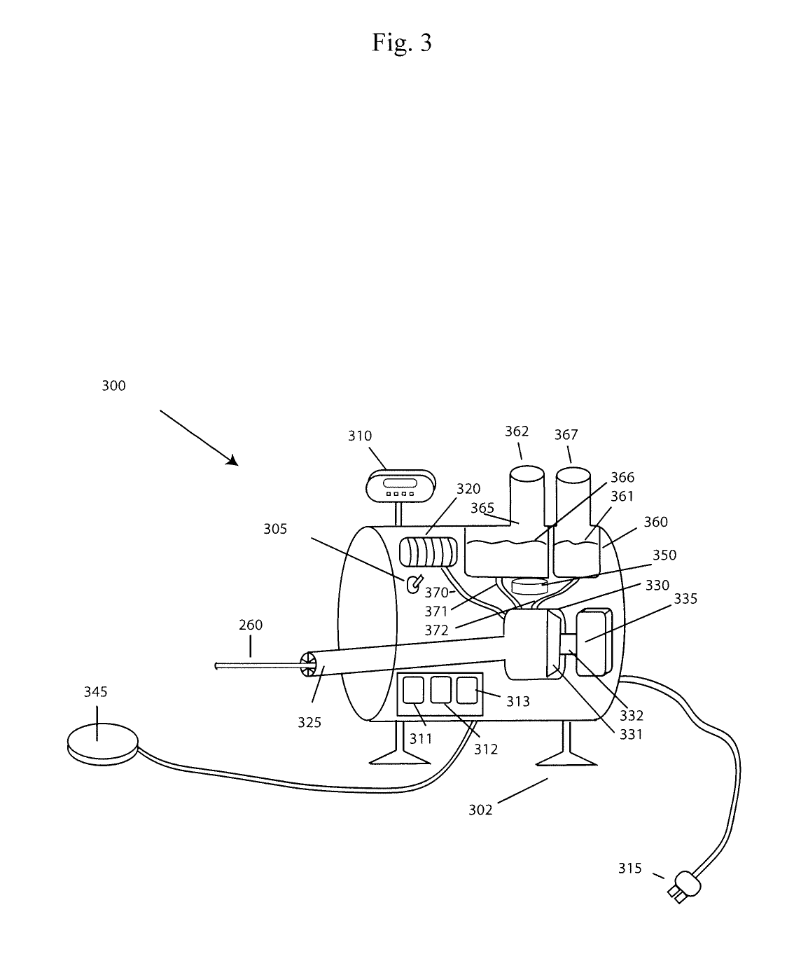

As shown in FIGS. 3A-3C, the fluid directing device 300 may contain a shell 301 for housing the components (see FIG. 1A), feet 302 for supporting the device 300 standby switch 305, user interface 310, microprocessor or control logic 311 and computer readable memory or storage media 312, timing circuit 313, a plug 315, air intake 320, fluid directing device connector 325, a pump 330 (which may feature a diaphragm 331 and/or piston 332), motor 335, actuator 345, tanning fluid warming element 350, cleaning fluid reservoir 360, and tanning fluid reservoir 365, cleaning fluid 361, and tanning fluid 366. The reservoirs may contain inlets 362 and 367. The air intake and reservoirs may contain ports (370-372) for providing a fluid connection to the pump 330. The user interface 310 may allow the user to interact with the fluid directing device 300 to instruct the fluid directing device 300 to execute preprogrammed instructions stored in the computer readable memory 312 and executed by microprocessor control logic 311. The actuator 345 may feature a wired connector or may use a wireless connection method to the fluid directing device 300.

To apply tanning solution to a user, a user may follow an assembly process 400, a tanning process 500, and a cleaning process 600. The following processes are exemplary and certain steps may be omitted, added, repeated, or performed in differing orders.

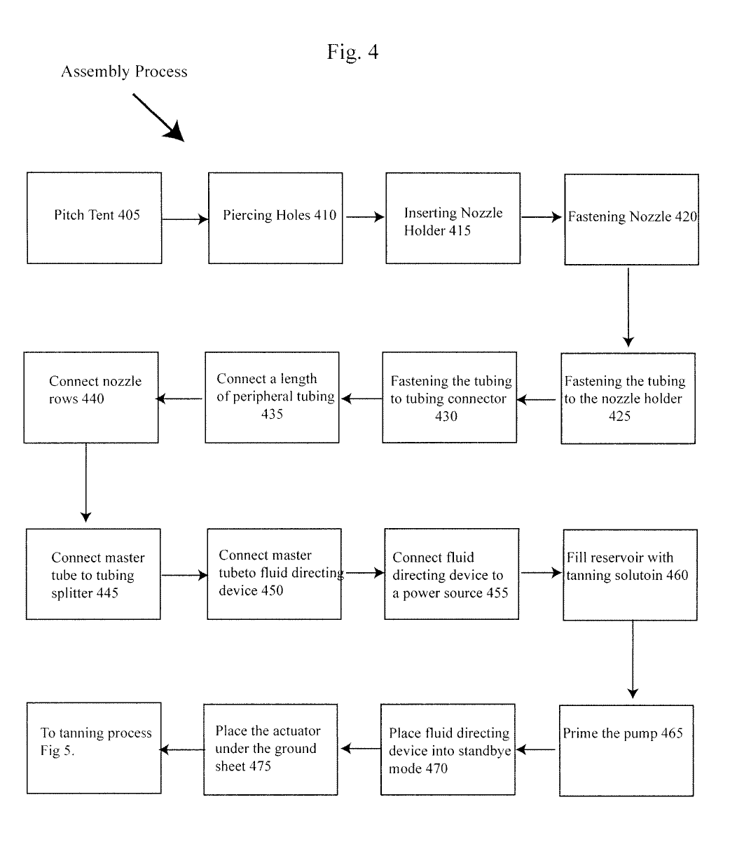

As shown in FIG. 4, the assembly process 400 features a user pitching the tent 405 apparatus 100, piercing 410 holes into the canopy using a punch, inserting 415 a nozzle holder so that nozzle holder surrounds the hole, fastening 420 (by screwing for example) a nozzle to the inside of the nozzle holder so that the nozzle will spray into the tent, fastening 425 the tubing connector to the nozzle holder, and fastening 430 the tubing to the tubing connector 230. Other nozzles and tubing may be connected in a series to create nozzle row 280. A nozzle row 280 contains at least three sets of nozzles 210A-210C, at least two lengths of peripheral tubing 240A-240B, three nozzle holders 220A-220C, and three tubing connectors 230A-230C. The tent apparatus may be designed to accommodate 1, 2, 3, 4, or more nozzle rows 280 to facilitate tanning of a user from multiple angles, or facilitate tanning of multiple people. The user may then connect 435 a length of peripheral tubing to a tubing connector 230 (such as the lower or upper most tubing connector) of the nozzle row 280 to the tubing splitter 250. The user may then connect 440 other nozzle rows 280B to the tubing splitter 250, so that all nozzle rows are attached at a central location such as the tube splitter 250, so that all nozzle rows 280A and 280B are fluidly connecting to a master tube 260. The master tube 260 may have a larger diameter than the peripheral tubing 240, and be connected on one side to the tubing splitter 445 and connected on the other side 450 to the fluid directing device.

The user may connect 455 the fluid directing device 300 to a power source such as an outlet or battery, fill 460 the reservoir 365 with tanning solution, prime 465 the pump, and place 470 the fluid directing device into the standby mode by flipping the standby switch. The user may place 475 the actuator (which can be a foot pedal and a wire—though wireless models can be used) underneath the groundsheet, but above the footprint. The groundsheet may have an indentation or formed contoured shape for receiving the actuator, and the groundsheet may be specially colored in the location of the actuator.

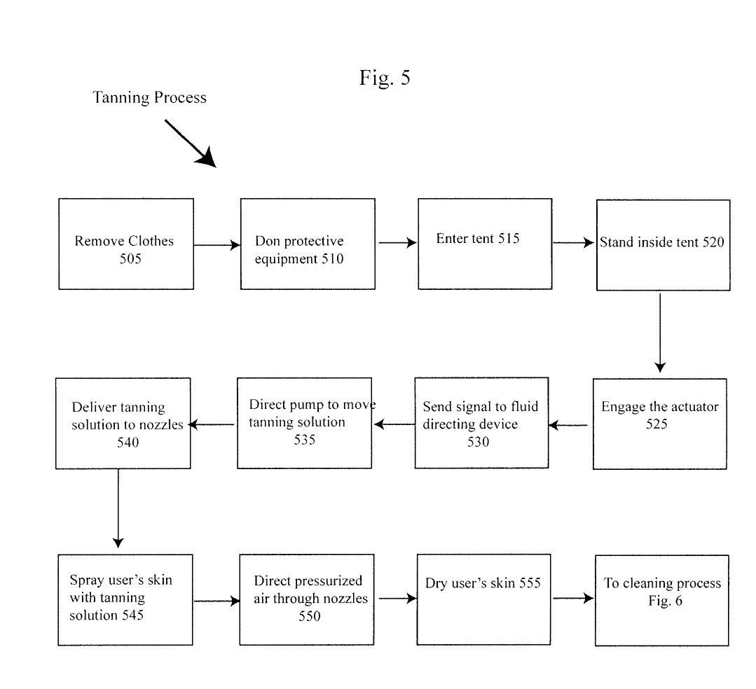

In FIG. 5, the tanning process, the user would remove 505 his or her clothes, don 510 protective optional equipment such as goggles, swim cap, and booties, and enter 515 the tent. The tent apparatus as illustrated has a front zippered entrance, such that the user can step through when unzipped. Once inside, the user would zip the entrance to essentially close the entrance, and stand 520 inside the tent. Using the actuator underneath the groundsheet of the tent, the user would engage 525 the actuator by, for example, stepping on it.

The actuator 345 may be a simple on/off switch, or it may also have additional settings that can be set (duration, spray pressure, tanning solution type (e.g. bronzing or clear) etc). Once engaged, the actuator 345 would send 530 a signal to the fluid directing device 300, which would cause the device to direct 535 the pump 330 to move tanning solution from the reservoir 360 through the pump 330, into the fluid directing device connector 325 into the master tube 260, wherein the tanning solution 361 will be delivered 540 to the nozzles 210, where it will be sprayed 545 and onto the user by way of the peripheral tubes 240. The fluid directing device 300 may also direct 550 pressurized air into the tent to help the tanning solution 351 dry 555 on the user's skin. The tanning fluid warming element 350 can raise the temperature of the tanning fluid so that it does not irritate the user's skin, as the pressure created by the fluid directing device 300 can make the apparent temperature of the tanning solution feel cold to the user.

In prior art spraying systems, the user often would have to change poses so that the tanning solution hits all surfaces, but in this system, omnidirectional nozzles (i.e. nozzles that surround the user on 2, 3, or 4 sides see FIG. 1) promote an even distribution of tanning solution so that user does not need to change poses while tanning.

The fluid directing system may propel tanning solution into the tent for 15 seconds-75 or more seconds. Typically 30-45 seconds will be sufficient. The timing operation for the fluid directing device 300 may be controlled via the timing circuit 313, and the timing circuit 313 may be separate, or integrated into the user interface 310 or actuator 345.

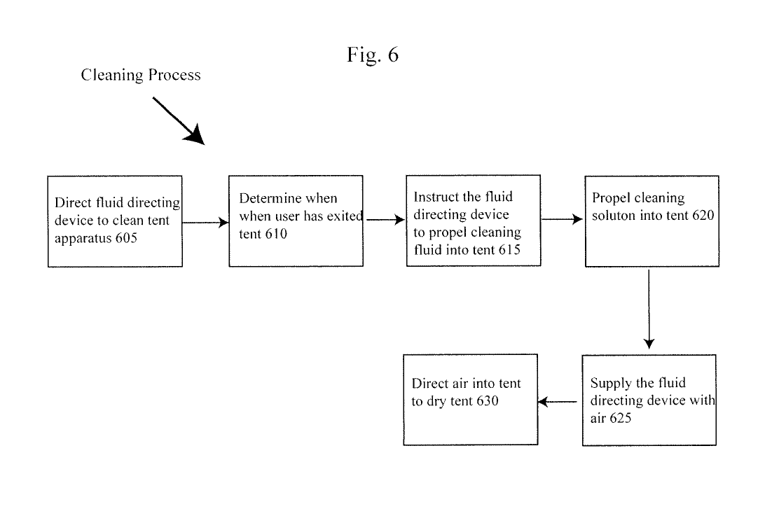

As shown in FIG. 6, the cleaning process 600 may be used to clean the tent apparatus 100 and nozzles 210 and tubes 240, etc. To do so, the fluid directing device 300 may contain a reservoir 360 for cleaning solution 361 (or may contain a connector to receive a bottle of cleaning solution.) The cleaning solution 361 can be water, alcohol, detergent, or other cleaning fluids. The user can direct 605 the fluid directing device 300 to clean the tent, by pushing 302 a button on the user interface 310 on the fluid directing device 300, or the cleaning process 600 can be automatically executed 303 after the user exits the tent. In that later configuration, the tent may contain a detecting apparatus 170, such as a proximity detector, motion detector, or a weight detector to determine 610 when the user has exited the tent. Once the detecting apparatus 170 has determined 610 that the user is out of the tent apparatus 100, the detecting apparatus 170 can instruct 615 the fluid directing device 300 to propel 620 cleaning solution 361 into the tent after a specified time and for a specified duration. The fluid directing device 300 can also propel 630 air into the tent apparatus 100 through the nozzles 210 to dry the tent apparatus 100 more quickly. The air intake 320 may supply 625 the fluid directing device with air.

1. A system for applying a tanning spray to a user comprising a tent apparatus, spray apparatus, and fluid directing apparatus:
a. said tent apparatus containing a canopy, a fly, a groundsheet, a footprint, an entrance, and supports for holding the canopy;
b. said spray apparatus containing a nozzle, a nozzle holder, tubing connector, peripheral tubing, tube splitter, master tube, and a fluid directing device connector, said peripheral tubing connecting several nozzles together and providing a fluid pathway to the tube splitter; said nozzle holder positioned in holes in the canopy and providing a mounting surface for the nozzles to be secured into, thereby fixing the nozzles to the canopy; said tubing connector connecting the nozzles to the peripheral tubes; and
c. said fluid directing device containing a user interface, microprocessor and computer readable memory, a pump, motor, actuator, tanning fluid reservoir, and tanning fluid;
d. wherein the pump and motor are configured to direct tanning solution from the tanning solution reservoir through the pump, peripheral tubing, and out of the nozzles thereby applying the tanning solution to the user.
